(19) United States
(12) Patent Application Publication
(10) Pub. No.: US 2011/0137305 A1
(43) Pub. Date: Jun. 9, 2011

(54) THERMAL NEUROABLATOR

(76) Inventor: Gregorio Hernandez Zendejas, Guadalajara (MX)

(21) Appl. No.: 12/631,853

(22) Filed: Dec. 6, 2009

(51) Int. Cl. A61B 18/04

(52) U.S. Cl. 606/30

(57) ABSTRACT

Thermal neuroablator (FIG. 1) for modulating the facial kinetics. A thermal needle tip (100) is provided for applying thermal energy to a facial nerve via the percutaneous route. A switch (108) for alternating between two or more circuit configurations connected to thermal needle tip (100). A power supply for providing power connected to switch (108).

### TECHNICAL FIELD

This application relates to the field of surgery and, more particularly, to minimally invasive surgery.

### CROSS-REFERENCE TO RELATED APPLICATIONS

Not applicable.

### FEDERALLY SPONSORED RESEARCH

Not applicable.

### SEQUENCE LISTING OR PROGRAM

Not applicable.

### BACKGROUND

The facial muscles are a group of striated muscles innervated by the facial nerve that, among other things, control facial kinetics. Modification of the facial kinetics is beneficial in certain medical conditions. A well-established procedure for modulating the facial kinetics involves the surgical excision of a portion of a facial muscle (selective myectomy). Selective myectomy is used to treat certain medical conditions such as facial paralysis, and migraine (Baker D C: Facial palsy. In McCarthy J G (Ed): Plastic Surgery Vol. 3 The Face. Philadelphia: W.B. Saunders Company, 1990, p 2306; Guyuron B, Kriegler J S, Davis J, Amini S B: Comprehensive surgical treatment of migraine headaches. Plast Reconstr Surg 115:1, 2005). Selective myectomy or myotomy is critical to achieve improved aesthetic results in aesthetic facial surgery (Abramo A C, Dorta A A: Selective Myotomy in Forehead Endoscopy. Plast Reconstr Surg 112:873, 2003). Nevertheless selective myectomy involves surgical and anesthetic risks.

In an attempt to avoid these surgical and anesthetic risks, minimally invasive methods for reduce the function of the facial muscles have been proposed. These minimally invasive methods involve the application of either energy or a substance through the skin. The application of either energy or a substance through the skin can be percutaneous or transcutaneous. Percutaneous pertains to any medical procedure where access to inner organs or other tissue is done via needle-puncture of the skin, rather than by using an “open” approach where inner organs or tissue are exposed. Transcutaneous pertains to any medical procedure where access to inner organs or other tissue is done via the unbroken skin.

Percutaneous, and transcutaneous minimally invasive methods for reduce the function of the facial muscles have been proposed. One of those methods involves percutaneous botulin toxin injections. Botulin toxin paralyses muscles, reducing the facial muscles function (Bulstrode N W, Grobbelaar A O: Long-term prospective follow-up of botulinum toxin treatment for facial rhytides. Aesth Plast Surg 26:356-359, 2002). However, botulin toxin injections effect lasts a few months.

Implants for continuous in vivo release of a neurotoxin over a period ranging from several days to a few years have been proposed (U.S. Pat. No. 6,383,509 B1; U.S. Pat. No. 6,506,399 B2; U.S. Pat. No. 6,312,708 B1; U.S. Pat. No. 6,585,993 B2; U.S. Pat. No. 6,306,423 B1). These implants can contain botulin toxin. Nevertheless if for some reason the botulin toxin contained in such implants is released faster than expected, poisoning caused by botulin toxin may issue.

Percutaneous denervation of facial muscles to modify the facial kinetics has been reported (Hernandez Zendejas G, Guerrerosantos J: Percutaneous selective radio-frequency neuroablation in plastic surgery. Aesth Plast Surg 18:41-48, 1994; Utley D S, Goode R L: Radiofrequency ablation of the nerve to the corrugator muscle for elimination of glabellar furrowing. Arch Facial Plast Surg 1:46, 1999; U.S. Pat. No. 6,139,545; US 2005/0283148 A1; US 2007/0060921 A1; US 2007/0167943 A1). This technique involves the destruction of nerves by electro fulguration (charring) of tissues using high-energy radio frequency energy.

However, long-term results can be unpredictable because denervated facial muscles undergo reinnervation (Baker D C: Facial palsy. In McCarthy J G (Ed): Plastic Surgery Vol. 3 The Face. Philadelphia: W.B. Saunders Company, 1990, p 2255). Denervation of facial muscles can be burdensome since facial muscles innervation is inconsistent (Schwember G, Rodriguez A: Anatomical surgical dissection of the extraparotid portion of the facial nerve. Plast Reconstr Surg 81:183, 1988; Caminer D M, Newman M I, Boyd J B: Angular nerve: New insights on innervation of the corrugator supercilii and procerus muscles. Plast Reconstr Aesth Surg 59:366, 2006).

Transcutaneous methods and devices for treatment of a muscle and nerve have been proposed (US 2007/0255342 A1). Such methods involve applying transcutaneous radio-frequency energy, and high-voltage current, which may create significant liability concerns.

Transcutaneous energy delivering for creating a lesion in facial muscles to modify the facial kinetics has been proposed (US 2007/0032784 A1; US 2008/0071255 A1). Nevertheless this technique is not muscle-specific and creates a lesion not only in muscle tissue, but also in contiguous vital structures such as nerves, arteries, veins, and lymphatics.

Percutaneous selective thermal ablation of facial muscles to modify the facial dynamics has been reported (Hernandez Zendejas G. Percutaneous Myoablation Utilizing a Low-Energy Probe for Corrugator Supercilii Muscles. Abstracts of the 35th International Annual Symposium of Plastic Surgery—Aesthetics 2009, pp 24, Nov. 11, 2009. Puerto Vallarta, Jalisco, Mexico). This technique achieves an 83.3% of good results. It is necessary to increase this percentage of good results. A combined procedure including thermal ablation of facial muscles and thermal neuroablation can increase this percentage.

Several patients can benefit from a combined procedure of thermal ablation of facial muscles and thermal neuroablation. Current devices for the performance of neuroablation produce an unnecessary charring of tissues using radio frequency energy, and are difficult to use (U.S. Pat. No. 6,139,545; US 2005/0283148 A1; US 2007/0060921 A1; US 2007/0167943 A1). Moreover, such devices involve the passage of high-intensity electrical current through the body, which may create significant liability concerns.

It is unnecessary to charring tissues to achieve a neuroablation. Some tissues such as nerves and vessels are very sensitive to heat. Even a low temperature (below 44° C.) will result in tissue death if the duration of exposure is prolonged. Between 44° C. and 51° C. the rate of cell destruction doubles with each degree rise in temperature. At above 70.0° C. the tissue destruction is instantaneous (Salisbury R E: Thermal Burns. In McCarthy J G (Ed): Plastic Surgery Vol. 1 General Principles. Philadelphia: W.B. Saunders Company, 1990, p 790).

Thermal ablation does not involve the passage of electrical current through the body. However, suitable devices to perform a percutaneous selective thermal neuroablation are not available. Some devices that could be used in minimally invasive surgery are impractical for use in percutaneous selective thermal neuroablation (U.S. Pat. No. 2,030,285; U.S. Pat. No. 3,234,356; U.S. Pat. No. 3,461,874; U.S. Pat. No. 3,613,682; U.S. Pat. No. 3,662,151; U.S. Pat. No. 3,886,944; U.S. Pat. No. 3,978,312; U.S. Pat. No. 4,108,181; U.S. Pat. No. 6,482,200 B2; U.S. Pat. No. 3,698,394; U.S. Pat. No. 6,824,555 B1; U.S. Pat. No. 6,872,203 B2)

### SUMMARY

Thermal neuroablator for modulating the facial kinetics. A thermal needle tip is provided for applying thermal energy to a facial nerve via the percutaneous route. A switch for alternating between two or more circuit configurations connected to the thermal needle tip. A power supply for providing power connected to the switch.

### BRIEF DESCRIPTION OF THE DRAWINGS

A complete understanding of the present invention may be obtained by reference to the accompanying drawings, when considered in conjunction with the subsequent, detailed description, in which:

FIG. 1 is an overall view of a thermal neuroablator of the first embodiment;

FIG. 2 is a perspective view of the thermal neuroablator shown in FIG. 1;

FIG. 3 is a schematic view of the thermal neuroablator shown in FIG. 1;

FIG. 4 is an enlarged partial view of the thermal neuroablator shown in FIG. 3;

FIG. 5 is a perspective view of the thermal needle tip shown in FIG. 2;

FIG. 6 is a perspective sectional view of the thermal needle tip shown in FIG. 5;

FIG. 7 is an enlarged view of the distal end of the thermal needle tip shown in FIG. 5;

FIG. 8 is an enlarged sectional view of the distal end of the thermal needle tip shown in FIG. 6;

FIG. 9A is a perspective view of an additional embodiment of the thermal needle tip;

FIG. 9B is a perspective view of an additional embodiment of the thermal needle tip;

FIG. 9C is a perspective view of an additional embodiment of the thermal needle tip;

FIG. 9D is a perspective view of an additional embodiment of the thermal needle tip;

FIG. 10 is a schematic view of a thermal neuroablation procedure;

FIG. 11 is a schematic view of a thermal neuroablation procedure;

FIG. 12 is a schematic view of a thermal neuroablation procedure;

FIG. 13A is a schematic view of a thermal neuroablation procedure;

FIG. 13B is a schematic view of a thermal neuroablation procedure;

FIG. 14A is a perspective view of an alternate embodiment of the thermal neuroablator;

FIG. 14B is a partial view of an alternate embodiment of the thermal neuroablator; and

FIG. 14C is a partial view of an alternate embodiment of the thermal neuroablator.

For purposes of clarity and brevity, like elements and components will bear the same designations and numbering throughout the Figures.

### GLOSSARY

Glossary of technical and medical terms.

Heat: A flow of energy between two objects or systems due to temperature difference between them.

Facial muscles. Group of striated muscles innervated by the facial nerve that, among other things, control facial expression.

Corrugator supercilii muscles: Skeletal muscles of the forehead that produce frowning and brow depression.

Corrugator supercilii muscle terminal motor nerve: The nerve that controls the corrugator supercilii muscle.

Thermal neuroablation: Devitalize a nerve by applying thermal energy via the percutaneous route (neuro=nerve: a prefix used in biology to denote nerve; ablation=erosion: removal of a part of biological tissue; to remove or decrease something by the process of ablation).

Thermal neuroablation procedure: Series of steps taken to accomplish a thermal neuroablation.

Thermal neuroablator: Device to perform a thermal neuroablation procedure.

### DETAILED DESCRIPTION

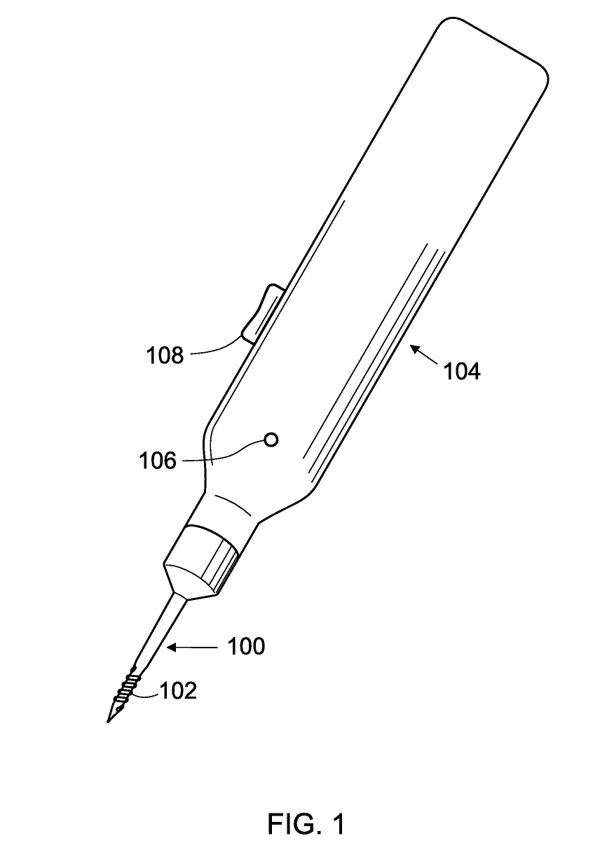

### First Embodiment—FIGS. 1, 2, 3, 4, 5, 6, 7, 8, 9A, 9B, 9C, 9D

A prototype of the thermal neuroablator was built, and tested by the inventor.

FIG. 1 shows an overall view of the thermal neuroablator of the first embodiment. The thermal neuroablator of FIG. 1 has a handle 104 and a detachable thermal needle tip 100. Handle 104 has a time indicator 106 and a switch 108. Thermal needle tip 100 has a resistive heating element 102.

Thermal needle tip 100 is provided for applying thermal energy to a facial nerve via the percutaneous route.

I contemplate that handle 104 of this embodiment is made of plastic, but others materials are also suitable.

Time indicator 106 is provided for measuring time of actual delivering of energy to thermal needle tip 100. I presently propose that time indicator 106 of this embodiment consists of a blinking LED with a 2.4 Hz blinking rate available from Super Bright LEDs Inc. St. Louis Mo., USA (www.superbrightleds.com). Notwithstanding others components for measuring or indicating time are also suitable, such as a clock, watch, stopwatch, etc.

Switch 108 is provided for alternating between two or more circuit configurations. I presently contemplate that switch 108 of this embodiment consists of a single pole single throw momentary (push-to-make) switch. However others components for alternating between two or more circuit configurations are also suitable, such as a microswitch, reed switch, tilt switch, transistor, relay, computer-controlled switching mechanism, optoelectronic mechanism, nanotechnology mechanism, molecular mechanism, etc.

Power supply 110 (not visible) is provided for supplying power to thermal needle tip 100. I presently propose that power supply 110 of this embodiment consists of three AA size 1.5 volt alkaline batteries. These three batteries are arranged to provide a total of 4.5 volts. Nevertheless other power sources are also suitable, such an electrochemical cell, thermoelectric power generator, etc.

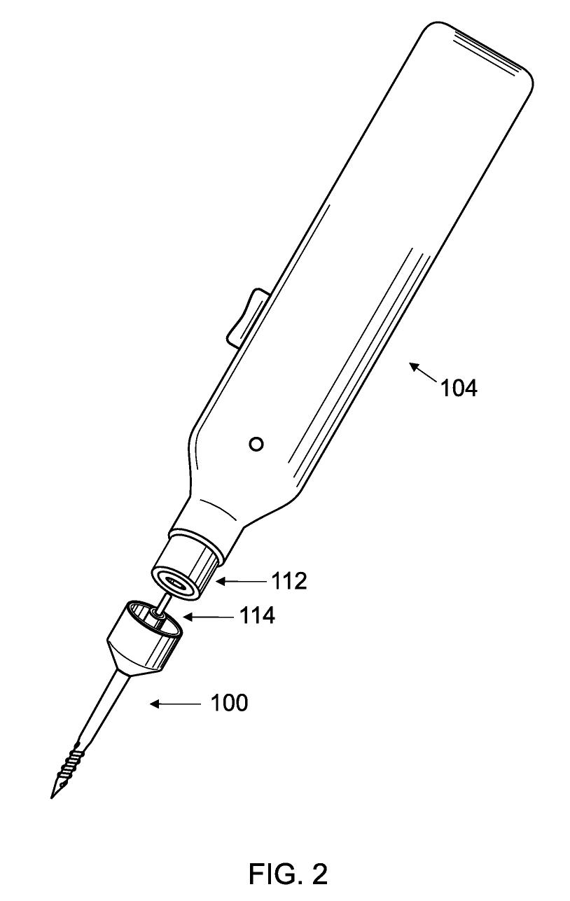

FIG. 2 shows a perspective view of the thermal neuroablator shown in FIG. 1. Thermal needle tip 100 is showing detached from Handle 104. Handle 104 has a coaxial jack 112. Thermal needle tip 100 has a coaxial plug 114.

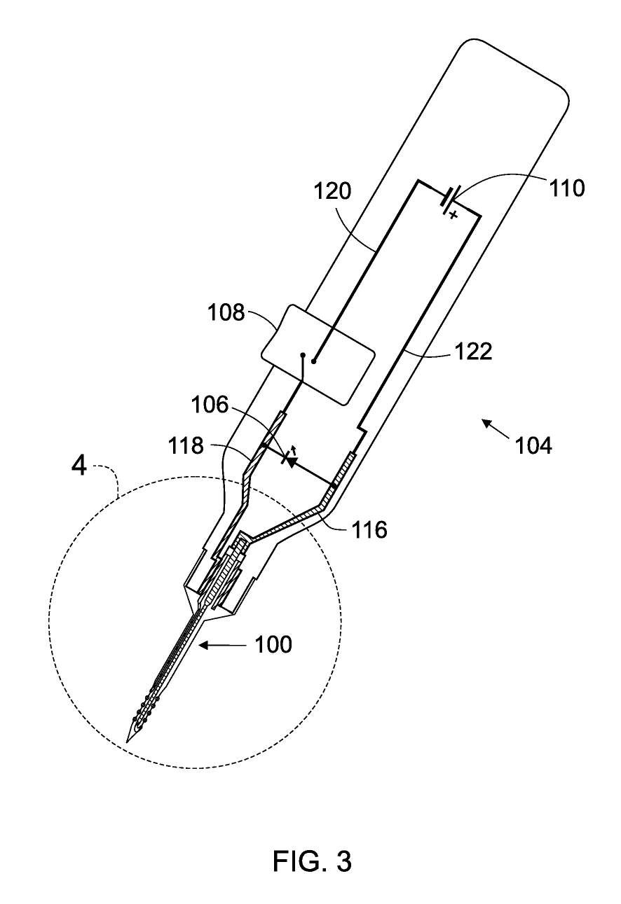

FIG. 3 shows a schematic view of the thermal neuroablator shown in FIG. 1. Handle 104 is attached to thermal needle tip 100.

Power supply 110 is connected by a negative wire 120 to switch 108 terminals. Power supply 110 is connected by a positive wire 122 to a central conductor 116.

Switch 108 is connected to a middle conductor 118.

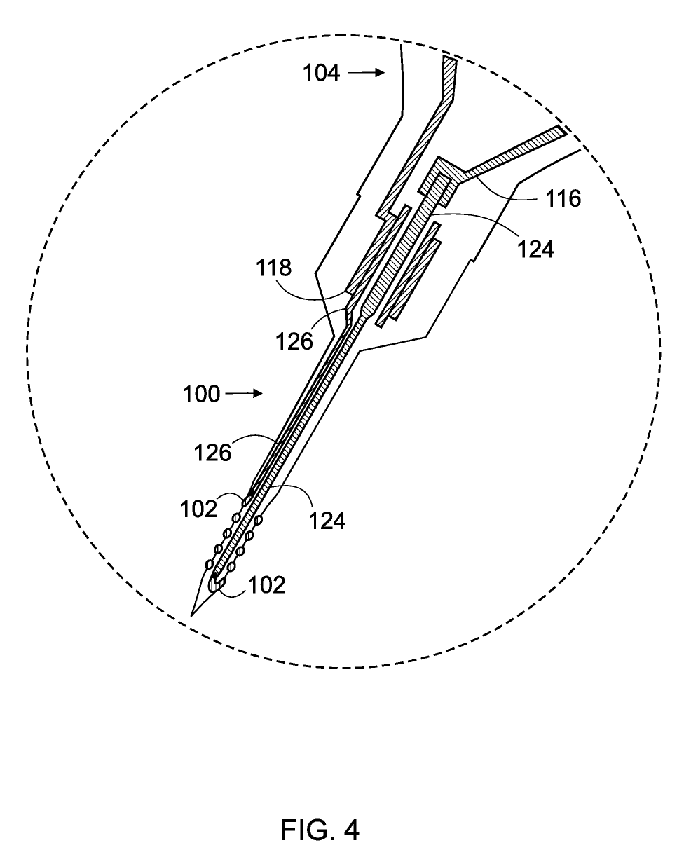

Time indicator 106 is connected to central conductor 116, and to middle conductor 118. Connection between handle 104 and thermal needle tip 100 is shown enlarged in FIG. 4 for clarity.

I contemplate that negative wire 120, and positive wire 122 are made of highly-conductive copper alloy available from Industrial Electric Wire & Cable, Inc. 5001 S. Towne Drive, New Berlin, Wis. 53151 (www.iewc.com), but others materials are also suitable.

I presently propose that central conductor 116, and middle conductor 118 are made of highly-conductive gold-plated copper, but others materials are also suitable.

FIG. 4 shows an enlarged partial view of the thermal neuroablator shown in FIG. 3. Handle 104 is attached to thermal needle tip 100. Central conductor 116 is connected by a central rod 124 to resistive heating element 102. Resistive heating element 102 is connected by a middle rod 126 to middle conductor 118.

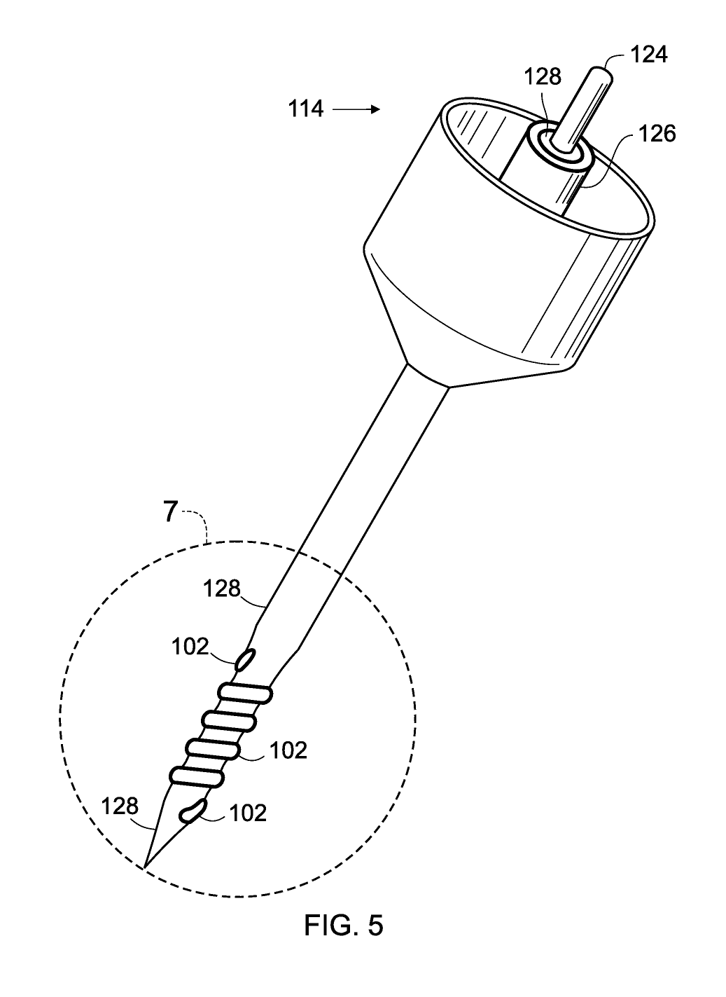

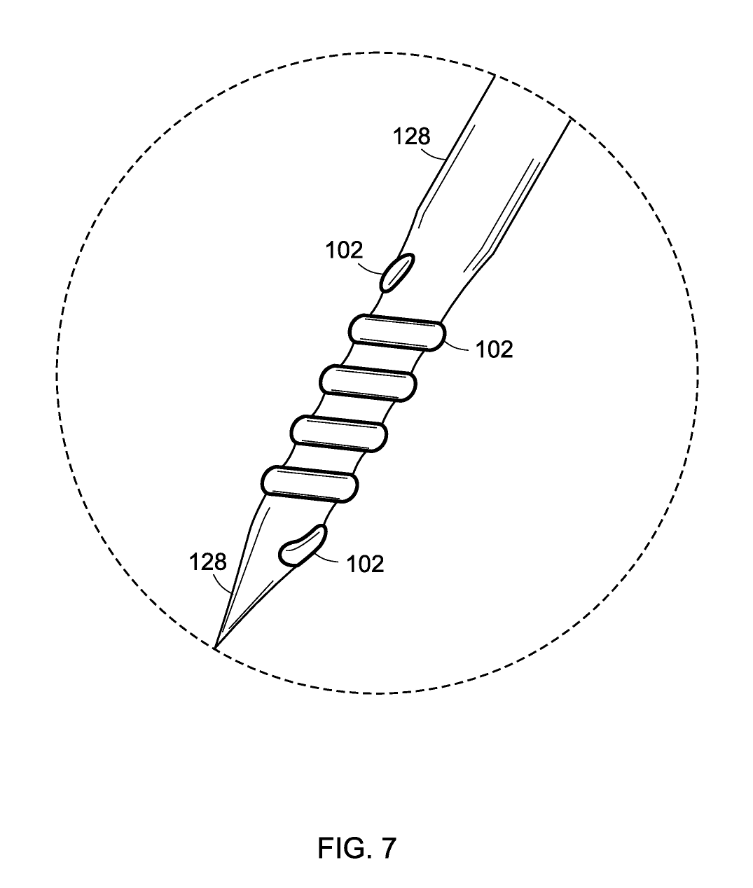

FIG. 5 shows a perspective view of thermal needle tip 100 shown in FIG. 2. Thermal needle tip 100 of FIG. 5 has coaxial plug 114, a thermal insulator matrix 128, and resistive heating element 102. Coaxial plug 114 comprises central rod 124, thermal insulator matrix 128, and middle rod 126. The distal end of thermal needle tip 100 is shown enlarged in FIG. 7 for clarity.

I presently propose that resistive heating element 102 have an overall resistance value of 6.3 ohms and be arranged in a 14 mm long coil of 40 loops. However it can have different resistant values, and different arrangements.

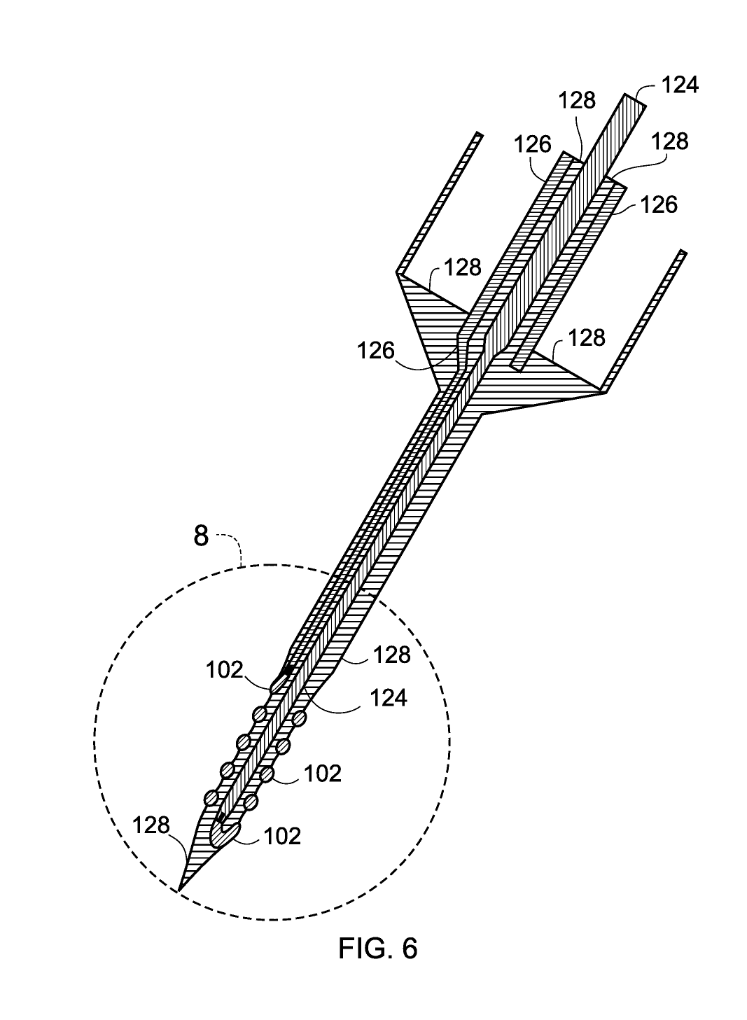

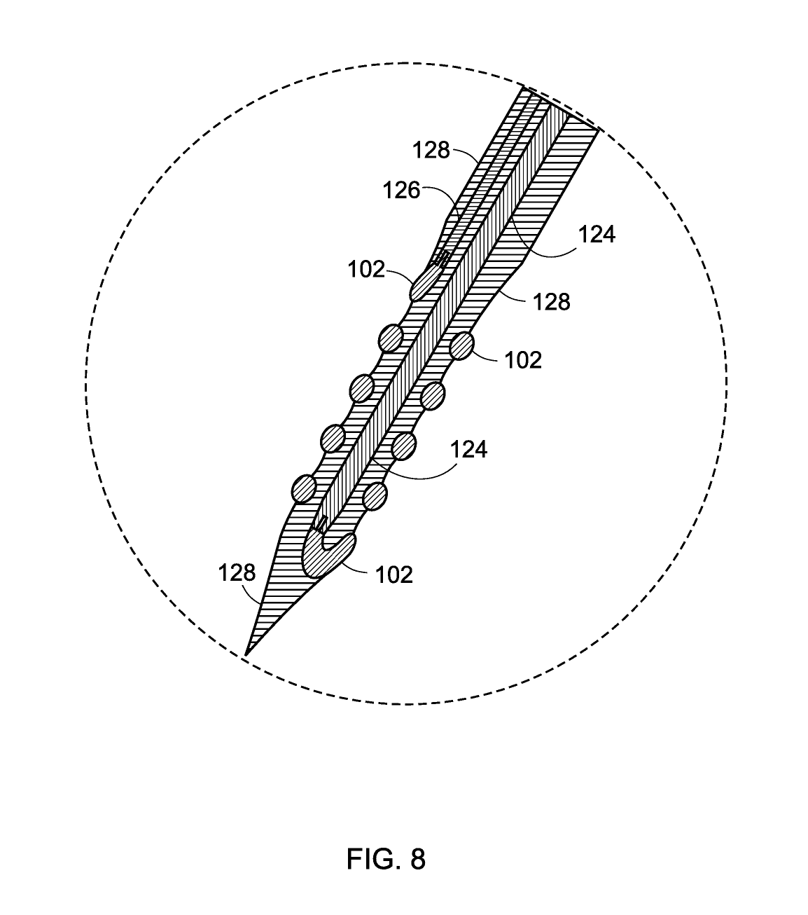

FIG. 6 shows a perspective sectional view of thermal needle tip 100 shown in FIG. 5. Central rod 124 is connected to resistive heating element 102. Resistive heating element 102 is connected to middle rod 126. Central rod 124, middle rod 126, and resistive heating element 102 are partially embedded in thermal insulator matrix 128. The distal end of thermal needle tip 100 is shown enlarged in FIG. 8 for clarity.

I presently contemplate that resistive heating element 102 is made of nickel-chromium resistance wire of 0.20 mm in diameter, available from WireTronic Inc. 19604 Mella Drive, Volcano, Calif. 95689-9786 USA (www.wiretron.com). However it can consist of any other material that can convert electrical energy into thermal energy.

I presently propose that central rod 124, and middle rod 126 are made of stainless steel, but others materials are also suitable.

I currently contemplate that thermal insulator matrix 128 is made of mullite available from McDanel Advanced Ceramic Technologies 510 9th Ave. Beaver Falls, Pa. 15010 (www.mcdanelceramics.com), but others materials are also suitable.

FIG. 7 shows an enlarged view of the distal end of thermal needle tip 100 shown in FIG. 5. Exterior surface of resistive heating element 102 is not covered by thermal insulator matrix 128.

FIG. 8 shows an enlarged sectional view of the distal end of thermal needle tip 100 shown in FIG. 6. Central rod 124 is connected to resistive heating element 102. Resistive heating element 102 is connected to middle rod 126. Central rod 124, middle rod 126, and resistive heating element 102 are partially embedded in thermal insulator matrix 128. Exterior surface of resistive heating element 102 is not covered by thermal insulator matrix 128.

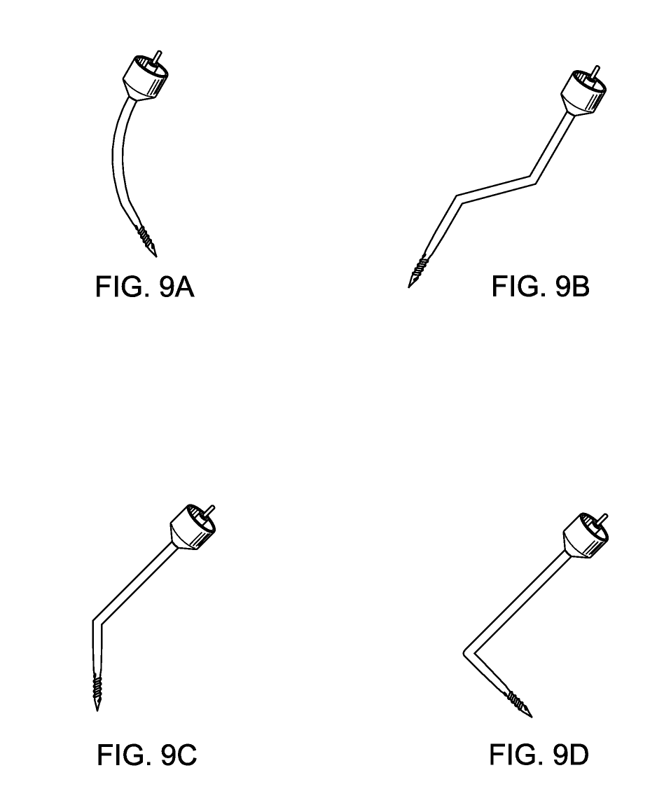

FIGS. 9A, 9B, 9C, and 9D shown additional embodiments of thermal needle tip 100, but others embodiments are also possible.

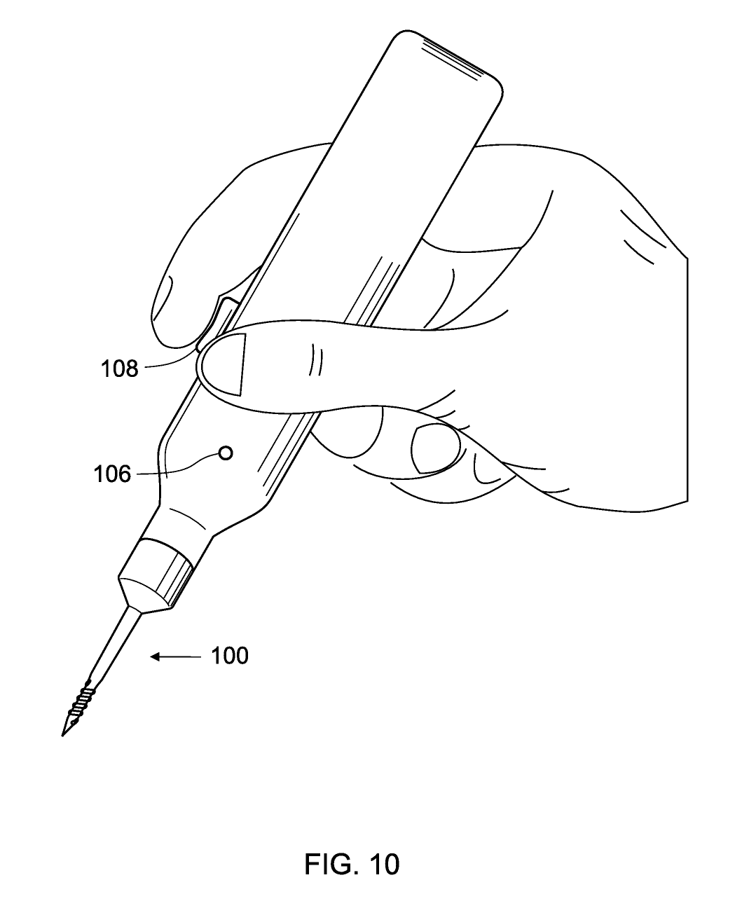

### Operation—First Embodiment—FIGS. 10, 11, 12, 13A, 13B

FIG. 10 shows an operational view of the thermal neuroablator shown in FIG. 1. When switch 108 is pressed, thermal needle tip 100 is energized. At the same time, time indicator 106 LED blinks at a 2.4 Hz blinking rate, allowing the physician to calculate the time thermal needle tip 100 remains energized.

Corrugator supercilii muscles are paired muscles that control frowning. Corrugator supercilii muscles terminal motor nerves control corrugator supercilii muscles movement. The patient is asked to frown to evaluate the degree of muscle activity.

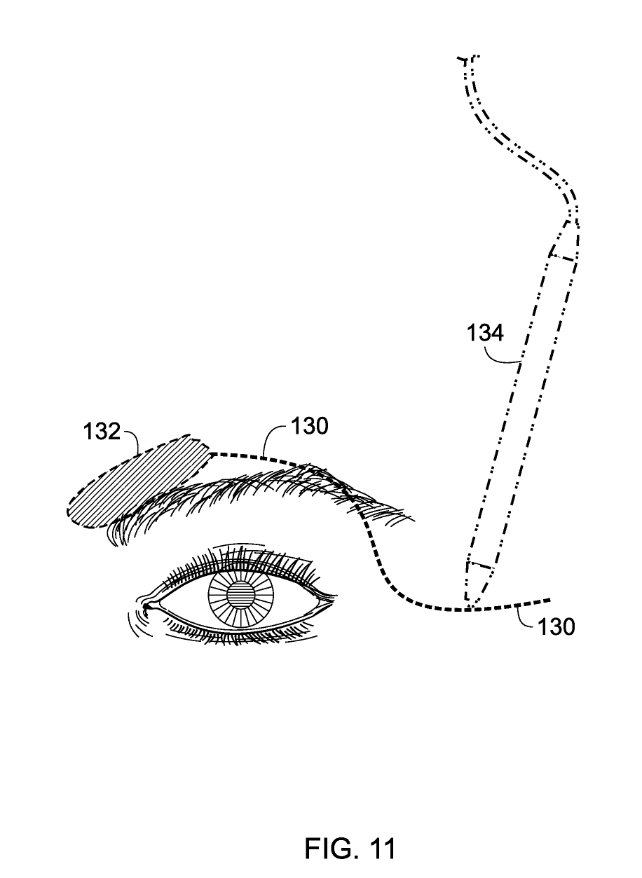

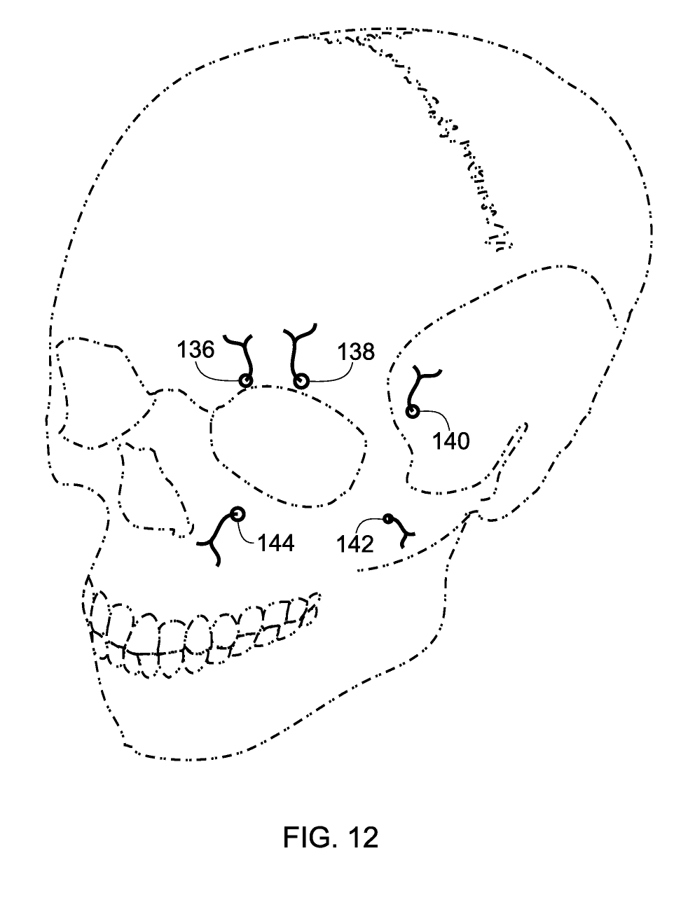

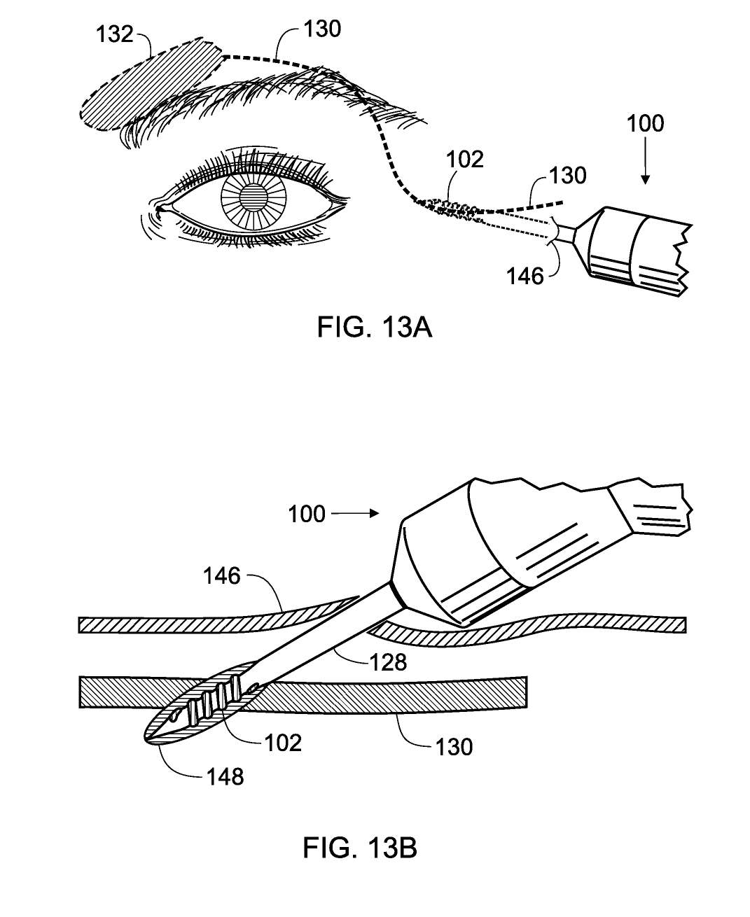

The thermal neuroablator can be used to perform a thermal neuroablation procedure on one or more facial nerves. The thermal neuroablation procedure is accomplished in three steps as follows:
(1) Surface nerve location (FIG. 11)
(2) Regional anesthesia procedure (FIG. 12)
(3) Thermal neuroablation (FIGS. 13A and 13B)

(1) Surface nerve location (FIG. 11): A TENS 134 (transcutaneous electrical nerve stimulator) is used to perform the surface nerve location.

I currently contemplate that surface nerve location is performed utilizing a TENS Stimuplex® Pen available from B. Braun Medical Inc. U.S. Corporate Headquarters 824 Twelfth Avenue, Bethlehem, Pa. 18018 (www.bbraunusa.com), but other TENS are also suitable, such as a Hilger™ facial nerve stimulator available from WR Medical Electronics Co. 123 North Second Street Stillwater, Minn. 55082 USA (www.wrmed.com).

Left side corrugator supercilii muscle terminal motor nerve 130 is selected to illustrate how to use the thermal neuroablator to perform a thermal neuroablation procedure. TENS 134 delivers electrical pulses over the face to depolarize subcutaneous left side corrugator supercilii muscle terminal motor nerve 130. Rhythmic contractions of left side corrugator supercilii muscle 132 are observed once TENS 134 is just over the subcutaneous trajectory of left side corrugator supercilii muscle terminal motor nerve 130. This trajectory is marked with indelible ink.

(2) Regional anesthesia procedure (FIG. 12): Supratrochlear nerve 136, supraorbital nerve 138, zygomaticotemporal nerve 140, zygomaticofacial nerve 142, and infraorbital nerve 144 blocks are performed with 2% lidocaine/1:200,000 epinephrine solution.

(3) Thermal neuroablation (FIGS. 13A and 13B): A dermal papule with 2% lidocaine/1: 200,000 epinephrine solution is made on skin 146. Then thermal needle tip 100 is inserted percutaneously and directed toward left side corrugator supercilii muscle terminal motor nerve 130 in such a way that resistive heating element 102 is in contact with left side corrugator supercilii muscle terminal motor nerve 130.

Next, switch 108 (not shown) is pressed for two seconds, energizing thermal needle tip 100. Tissue in contact with resistive heating element 102 increases its temperature up to 70.0° C. This results in a well-demarcated tissue ablated area 148. A segment of left side corrugator supercilii muscle terminal motor nerve 130 is included in this tissue ablated area 148.

Thermal insulator matrix 128 limits the conduction of the heat generated by resistive heating element 102. The temperature of the portion of thermal needle tip 100 in contact with skin 146 is 37.5° C., therefore skin 146 is undamaged.

Next, thermal needle tip 100 is withdrew from the patient, and he or she is asked to frown to determine the degree of reduction of left side corrugator supercilii muscle 132 activity. To avoid burns at the insertion site, it is very important to allow thermal needle tip 100 to cool-down, waiting at least 15 seconds before withdrawing thermal needle tip 100.

Complete thermal neuroablation is corroborated asking the patient to frown. He or she should be unable to frown as forcefully as usual.

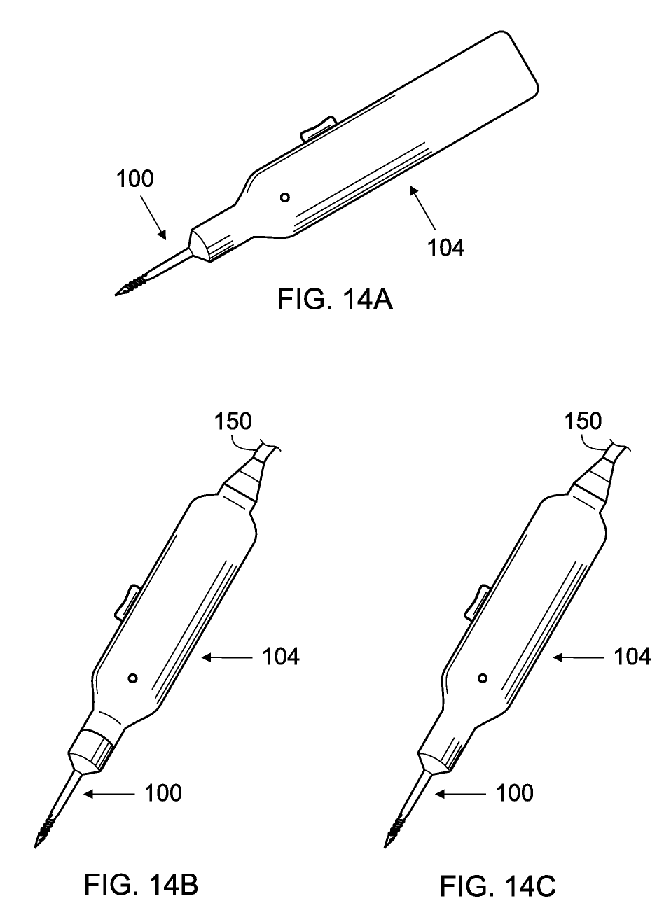

### Description—Second Embodiment—FIG. 14A

FIG. 14A shows a perspective view of the thermal neuroablator of the second embodiment. This embodiment is similar to the thermal neuroablator of FIG. 1. However thermal neuroablator of FIG. 14A comprises a handle 104 with a non-detachable thermal needle tip 100.

### Operation—Second Embodiment

The operation of the thermal neuroablator of FIG. 14A is similar to the operation of the thermal neuroablator of FIG. 1.

### Description—Third Embodiment—FIG. 14B

FIG. 14B shows a partial view of the thermal neuroablator of the third embodiment. This embodiment is similar to the thermal neuroablator of FIG. 1. However thermal neuroablator of FIG. 14B comprises a handle 104 connected by power cable 150 to an external power source (not shown), and a detachable thermal needle tip 100. I presently propose that the external power source of this embodiment consists of a 2 Amps regulated DC power source with an output of 4.5 volt DC. Nevertheless other external power sources are also suitable, such as a battery, electrochemical cell, thermoelectric power generator, etc.

### Operation—Third Embodiment

The operation of the thermal neuroablator of FIG. 14B is similar to the operation of the thermal neuroablator of FIG. 1.

### Description—Fourth Embodiment—FIG. 14C

FIG. 14C shows a partial view of the thermal neuroablator of the fourth embodiment. This embodiment is similar to the thermal neuroablator of FIG. 1. However thermal neuroablator of FIG. 14C comprises a handle 104 connected by power cable 150 to an external power source (not shown), and a non-detachable thermal needle tip 100. I presently propose that the external power source of this embodiment consists of a 2 Amps regulated DC power source with an output of 4.5 volt DC. Nevertheless other external power sources are also suitable, such as a battery, electrochemical cell, thermoelectric power generator, etc.

### Operation—Fourth Embodiment

The operation of the thermal neuroablator of FIG. 14C is similar to the operation of the thermal neuroablator of FIG. 1.

### Advantages

From the description above, a number of advantages of some embodiments of the thermal neuroablator become evident:

(a) Safety. The thermal neuroablator devitalizes a facial nerve by increasing its temperature up to 70.0° C., thus avoiding the need to charring tissues to produce the desired effect. No electrical current pass through the patient's body, thus minimizing the risk of liability issues. The whole procedure is performed under local anesthesia. Mayor surgical and anesthetic risks are avoided. Disposable, sterilized tips can be manufactured at a very low cost.

(b) Extensive clinical applications. The thermal neuroablator can be employed for the treatment of migraine, facial paralysis, spasmodic torticollis, blepharospasm, aesthetic enhancement, etc.

(c) Ease of use. The thermal neuroablator is easier to operate than radio-frequency ablation devices. The thermal neuroablator simple LED visual indicator is easy to read.

(d) Easy of production. The thermal neuroablator omits a built-in nerve stimulator found in prior nerve ablation devices, without loss of capability. The thermal neuroablator requires minimal engineering for manufacturing. The thermal neuroablator components are inexpensive and ready available. The thermal neuroablator does not require sophisticated production facilities.

### CONCLUSION, RAMIFICATIONS, AND SCOPE

While the above description contains many specificities, these should not be constructed as limitations on the scope of any embodiment, but as exemplifications of the presently embodiments thereof. Many other ramifications and variations are possible within the reachings of the various embodiments.

For example, thermal insulator matrix 128 can be eliminated. A highly skilled physician could perform a thermal neuroablation procedure using a thermal neuroablator without thermal insulator matrix 128. Thermal insulator matrix 128 can be replaced by a combination of a small skin incision and a small surgical retractor in order to avoid skin burns.

Thermal needle tip 100 can be modified to deliver other type of thermal energy (i.e. ultrasound, friction-mechanical, shock waves, micro shock waves, microwaves, laser, light, plasma, ionization, radiation, etc). Coaxial plug 114 can be replaced by a multi-axial connector. Coaxial jack 112 can be replaced by a multi-axial connector. Switch 108 can be replaced by an external switch (i.e. foot switch, etc.).

Thus the scope of the embodiments should be determined by the appended claims and their legal equivalents, and not by the examples given.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A thermal neuroablator for modulating the facial kinetics, comprising:
means for applying thermal energy to a facial nerve via the percutaneous route;
means for alternating between two or more circuit configurations, operably connected to said means for applying thermal energy to a facial nerve via the percutaneous route; and
means for providing power, operably connected to said means for alternating between two or more circuit configurations.

2. The thermal neuroablator in accordance with claim 1, wherein said means for applying thermal energy to a facial nerve via the percutaneous route comprises a thermal needle tip.

3. The thermal neuroablator in accordance with claim 1, wherein said means for alternating between two or more circuit configurations comprises a switch.

4. The thermal neuroablator in accordance with claim 1, wherein said means for providing power comprises a power supply.

5. A thermal neuroablator for modulating the facial kinetics, comprising:
a thermal needle tip, for applying thermal energy to a facial nerve via the percutaneous route;
a switch, for alternating between two or more circuit configurations, operably connected to said thermal needle tip; and
a power supply, for providing power, operably connected to said switch.

6. A thermal neuroablator for modulating the facial kinetics, comprising:
a thermal needle tip, for applying thermal energy to a facial nerve via the percutaneous route;
a switch, for alternating between two or more circuit configurations, operably connected to said thermal needle tip;
a power supply, for providing power, operably connected to said switch; and
a thermal insulator matrix, for providing thermal insulation, partially embedded to said thermal needle tip.
